(19) United States
(12) Patent Application Publication
(10) Pub. No.: US 2004/0167423 A1
(43) Pub. Date: Aug. 26, 2004

(54) RXc graph and RXc Z-score graph methods

(76) Inventors: Luana Pillon, New York, NY (US); Antonio Piccoli, Padova (IT)

Correspondence Address: QUINE INTELLECTUAL PROPERTY LAW GROUP, P.C., P O BOX 458, ALAMEDA, CA, 94501, US

(21) Appl. No.: 10/740,911

(22) Filed: Dec. 18, 2003

Related U.S. Application Data
(60) Provisional application No. 60/436,099, filed on Dec. 20, 2002.
(60) Provisional application No. 60/443,588, filed on Jan. 29, 2003.

(51) Int. Cl. A61B 5/053

(52) U.S. Cl. 600/547

(57) ABSTRACT

Methods and systems for displaying dimensionless vectors for bio-electric impedance measurements are provided. Systems and methods for displaying information as an RXc score graph are provided. Methods and systems for plotting Z(Xc) versus Z(R) for one or more patient or patient population and viewing the resulting Z(Xc)/Z(R) graph are provided.

### CROSS REFERENCE TO RELATED APPLICATIONS

This application is a non-provisional regular utility patent application of U.S. Ser. No. 60/436,099 to Luana Pillon, M.D., and Antonio Piccoli, M.D., entitled THE RXc GRAPH AND RXc Z-SCORE GRAPH METHODS, filed Dec. 20, 2002 and U.S. Ser. No. 60/`443,588 to Luana Pillon, M.D., and Antonio Piccoli, M.D., entitled THE RXc GRAPH AND RXc Z-SCORE GRAPH METHODS, filed Jan. 29, 2003. The subject application claims priority to and benefit of each of these prior provisional patent applications, each of which are incorporated by reference in their entirety for all purposes.

### COPYRIGHT NOTIFICATION

This document may contain material which is subject to copyright protection. All rights in such material are reserved.

### FIELD OF THE INVENTION

This invention is in the field of bioelectrical impedance measurements and graphical methods of assessing such measurements. Impedance vector distribution by sex, race, body mass index, and age in the United States, using standard reference intervals as bivariate Z scores is described.

### BACKGROUND OF THE INVENTION

Impedance is a function of capacitive reactance (Xc) and resistance (R). Impedance correlates with fluid balance in patients in a variety of contexts. In this respect, Bioelectrical impedance analysis (BIA) is a general diagnostic method that utilizes conductive properties of a body's tissues, cells, and fluids. Xc and R are typically measured during BIA using an impedance plethysmograph. The plethysmograph uses a constant current source producing a low-voltage electrical signal, (e.g., 800 micro-amps at a high frequency), e.g., at about 50 KHz, to set up an electrical signal in body (or a segment of the body), typically using a pair of surface EKG-type electrodes.

BIA is diagnostic of many different disease conditions. Because of this, measurements of whole-body bioelectrical impedance were collected in the last National Health and Nutrition Examination Survey (NHANES III), which was designed as a 6-year survey (1988 to 1994) to examine a nationally representative sample of the US civilian non-institutionalized population.[1, 2, 3, 4 and 5].

These surveys documented a marked increase in the prevalence of overweight and obesity in the US population that occurred primarily between NHANES II (1976 to 1980) and NHANES III (1988 to 1994).[1, 2 and 6] The body mass index (BMI; weight divided by the square of the height, kg/m2) was used to categorize body weight as low (<19 kg, underweight), acceptable (19 to 25 kg, healthy weight), or high (>25 kg, overweight).[7, 8, 9 and 10] Only 3.7% of American adults, aged 20 to 74 y, were considered underweight. Approximately 41.4% had a healthy weight. Among 54.9% of American adults who were overweight, 32.6% were further classified as preobese (25 BMI<30 kg/m2), 14.3% as class I obese (30 BMI<35 kg/m2), 5.2% as class II obese (35 BMI<40 kg/m2), and 2.8% as class III obese (BMI>40 kg/m2).[2 and 6] The greater prevalence of overweight was due to increases in the prevalence of obesity (BMI<30 kg/m2), whereas the prevalence of preobesity showed little or no change.[1, 2 and 6] Trends were similar for all age, sex, and race or ethnic groups. [2] Therefore, any reference population representative of most adult US subjects (74%) should include people with 19 BMI<30 kg/m2.

The National Center for Health Statistics measured the two impedance vector components, (resistance (R) and reactance (Xc)), in the NHANES III population but elected to archive results until valid, cross-validated prediction equations became available.[5]

Body impedance is represented by the impedance vector and is a combination of R (i.e., the opposition to flow of an alternating current through intra- and extracellular ionic solutions) and Xc (i.e., the reactance resulting from the capacitive character of cell membranes and tissue interfaces) across tissues.[11 and 12] Alternatively, the impedance vector can be expressed with a magnitude |Z|=.(R²+Xc²) and a phase angle (=arctan Xc/R). In simple biological conductors without cells (e.g., saline, urine, and ultrafiltrate), no Xc value can be measured; therefore, the impedance vector becomes an individual R value. Impedance is a measurable property of electrical ionic conduction of soft tissues. Lean soft tissues contribute more than fat soft tissues to impedance magnitude because adipocyte droplets (anhydrous triacylglycerols) are poor conductors. Contribution of bone is negligible (meaning it is a non-conductor) in the current range of biomedical analyzers. The portability, low cost, and non-invasive nature of impedance technology allow for clinical routine, bedside, single or repeated measurements.

Regression analysis has been used to derive empiric equations with the use of impedance values to predict measured body compartment volumes (meaning the criterion method's measurements). Many prediction equations of conventional bioelectrical impedance analysis (BIA) have been available in the literature since the 1980s[11] and several equations are recommended for estimating body compartments, like total body water, fat-free mass, and fat mass, particularly in white, adult, and healthy populations. [12] Recommended prediction equations, derived in healthy people and based on two- or multicomponent models, in cross-validation studies, have reached very high correlation coefficients (>0.97) and small prediction errors (standard error of the estimate) of 1.4 to 2.0 kg for total body water, 2.0 to 3.3 kg for fat-free mass, and 3.3 to 5.0% for fat mass. [12] Whether these same equations are applicable to disease states is open to discussion.

Nevertheless, the attractiveness of BIA lies in its potential as a stand-alone procedure free from anthropometry,[5] particularly in the clinical setting, where comparisons of the impedance vector of an individual patient with reference values are more useful than equations predicting average body compartments. Further, analysis of direct impedance measurement is not influenced by sampling error of regression coefficients or limitations in the accuracy of the criterion method. It has been shown that vector BIA, a pattern analysis of direct impedance measurements R and Xc from one subject, plotted as bivariate vectors on the race- and sex-specific tolerance intervals of the reference population (this is referred to as the “RXc graph method”), [13 and 14] is clinically useful in monitoring hydration in white adult populations of healthy subjects, [15] renal patients with altered hydration or undergoing chronic hemodialysis, [13 and 16] critical care patients, [17] and obese subjects with stable or changing weight. [13 and 18] The general validity of the method, however, should be better assessed by further clarifying the boundaries of the range of vectors that represent the normal variation in humans as compared with overt clinical conditions. [19]

In the literature, several reports have indicated the usefulness of statistical analysis of direct impedance measurements in uremic patients. In a multiracial hemodialysis population from the United States, univariate distribution (percentiles) of R and Xc components were calculated.[20] Further, an increased risk of mortality and morbidity has been documented in hemodialysis patients with a smaller phase angle [21] or a lower Xc value, [22] respectively. However, comparisons of phase angle or Xc vector component should be made between homogeneous groups of patients with the same sex and race, with comparable BMIs and vector magnitudes. [16 and 23] Misinterpretation of impedance readings in the clinical setting can be prevented by comparing both vector components (R and Xc) with a sex-, race-, and BMI-specific reference vector distribution. [13, 14, 15, 16, 17, 18, 24 and 25].

The present invention overcomes these difficulties, e.g., by providing a novel reference RXc-score graph (a bivariate Z score graph) that can be used with any analyzer in any population after transformation of impedance vector readings into standardized vector deviates (departures from the mean as a multiple of the standard deviation). These and many additional features will be apparent upon review of the following.

### SUMMARY OF THE INVENTION

The present invention provides methods and systems that use a dimensionless graph (e.g., a bivariate Z score graph) for diagnostic and prognostic analysis of patients and patient populations. The graph can be based upon whole body BIA or segmental (partial body) BIA and can be performed on any platform, e.g., by performing BIA at any frequency or set of frequencies used by the relevant platform.

Accordingly, in a first aspect, the invention provides methods of bioelectrical impedance vector analysis. In the methods, R and Xc are determined for a patient. R and Xc are converted into dimensionless numbers, and the resulting dimensionless numbers are plotted on a graph. In the methods, R and Xc are typically converted into dimensionless numbers by subtracting a population mean for R and Xc, respectively, from R and Xc, respectively, and dividing the resulting number by the standard deviation for R and Xc, respectively, in the population. In this case, the graph of standardized dimensionless numbers is an RXc Z-score graph. Thus, methods of bioelectrical impedance vector analysis that include determining Z(R) and Z(Xc) for a patient and plotting Z(R) and Z(Xc) on an RXc Z-score graph are provided.

In a related class of graphically performed bioelectrical impedance vector analysis methods, Z(Xc) versus Z(R) is plotted for one or more patient or patient population. The resulting Z(Xc)/Z(R) graph is then viewed. Typically, Z(Xc) versus Z(R) is plotted using a computer and the results are viewed on an appropriate user viewable (e.g., graphical or printed) display.

Similarly, methods of bioelectrical impedance vector analysis are provided in which a dimensionless measurement for R and a dimensionless measurement for Xc are determined, and the resulting dimensionless measurement for R is plotted against the dimensionless measurement for Xc. These can include Z(Xc) versus Z(R) as in the previous method. Here again, the results are typically plotted using a computer and viewed on an appropriate user viewable (e.g., graphical or printed) display.

In any of these methods, Z(R) and Z(Xc) are typically determined by taking impedance readings for R and Xc for the patient (these can be whole body readings, or can be readings for body segments). A mean value for R and Xc, respectively, for a reference population, are subtracted from the impedance readings R and Xc, respectively. The resulting values are divided by the standard deviation for R and Xc, respectively, in the reference population.

R and Xc are typically measured on an impedance plethysmograph (which can be a single frequency plethysmograph, or a multi-frequency plethysmograph). Typically, the magnitude of the impedance is equal to the square root of (R²+Xc²) and the impedance has a phase angle equal to arctan Xc/R and a length equal to the square root of (R²+Xc²).

The RXc Z-score graph that is produced in any of the methods can include one or more ellipse showing deviation from an absolute mean for a patient population. For example, the RXc Z-score graph can include three tolerance ellipses showing deviation from an absolute mean for a patient population at 50%, 75% and 95% deviation from mean. Placement of Z(R) and Z(Xc) for a patient of interest on the graph provides diagnostic information for the patient. For example, plotting Z(R) and Z(Xc) on the RXc Z-score graph provides an indication of whether the patient has more or less soft tissue or is more or less hydrated than a mean for a reference population. Similarly, placement on the graph is correlated to leanness, obesity, dehydration, anasarca, whether the patient is athletic, whether the patient is cachectic, whether the patient is normal, and/or any combination thereof. All of the Z(R) and Z(Xc) are determined for the patient or population by taking impedance readings for R and Xc for the patient or the population and subtracting a mean value for R and Xc, respectively, for a reference population, from the impedance readings R and Xc, respectively, and dividing the resulting values by the standard deviation for R and Xc, respectively, in the reference population.

Systems and/or kits for performing the above methods are also a feature of the invention. Thus, the invention comprises a bioelectrical impedance vector analysis system that includes a graph of a measure of Xc versus a measure of R, wherein Xc and R are dimensionless, and a user-viewable display displaying the graph. For example, the graph can be an RXc Z-score graph, e.g., generated by or embodied in system software. Thus, the invention provides a bioelectrical impedance vector analysis system comprising an RXc Z-score graph and a user-viewable display displaying the RXc Z-score graph. In either case, the system can include a computer that includes system software correlating the relevant graph to whether a patient coupled to the system is lean, obese, suffering from dehydration, suffering from anasarca, athletic, cachectic, and/or normal (or any other condition noted herein). The system optionally includes other features for obtaining appropriate measurements, such as electrical leads configured to be placed in contact with the skin of a patient, plethesmographs, etc.

### BRIEF DESCRIPTION OF THE FIGURES

FIG. 1 is an RXc graph showing bioelectrical impedance vector analysis (BIVA) patterns.

FIG. 2 is an RXc-score graph.

FIG. 3, panels A-C provide 3 example graphs: A. A plot of one point vector for every subject on the tolerance ellipses (RX point graph). B. A Plot of a sequence of point vectors for one subject on the tolerance ellipses (RXc path graph). C. A Plot of one bivariate Z-score vector for every subject on the reference RXc-score graph with three universal, tolerance ellipses (50%, 75%, and 95%).

FIG. 4, Panels A-B provide graphs of paired and unpaired data analysis. Panel A is a graph of unpaired data; Panel B is a graph of paired data.

FIG. 5 is a an RXc-Z score graph of several mean vector scores obtained from the literature after transformation of original impedance measurements into standard deviates with respect to the mean and SD of their reference populations.

FIG. 6 is a schematic system of the invention comprising a plethesmograph, leads to a patient and displays for an RXc-Z score graph.

### DEFINITIONS

The “RXc-Z score graph” is a graph of dimensionless numbers that are derived from R and Xc. The evaluation of an individual impedance measurement (obtained from any subject with any analyzer) with respect to a reference population (healthy representative people, same analyzer) can be generalized by transforming original, bivariate, impedance measurements into bivariate Z scores (where Z is a shortening for the standardized normal deviate and, in this context, the impedance vector score). Z scores are pure numbers (statistical scores with zero mean and unit SD) that are calculated for each subject, e.g., as the deviation of R/H and Xc/(HΩ/m) from their mean and divided by their SD. The bivariate distribution of Z scores can be represented with a probability graph, namely the RXc-score graph. Tolerance ellipses of Z scores, plotted as the RXc-score graph, preserve the same shape as the ellipses for the same percentiles on the original RXc graph (due to the same correlation coefficient) but have unique, fixed slopes for major (45°) and minor (−45°) axes [33].

For an “RXc graph” the parameters (slope and length of semiaxes) of the normal, bivariate confidence and tolerance intervals (ellipses) are calculated by following methods described elsewhere[13, 31, 32 and 33] and discussed in more detail below. Tolerance ellipses of an RXc graph are the bivariate percentiles directly indicating on the graph the probability that an individual vector falls at a given distance from an observed mean vector of a reference population (obtained with a same calibrated analyzer). Separate 95% confidence ellipses indicated a statistically significant difference (P<0.05) between mean vector positions on the R-Xc plane (which is equivalent to a significant Hotelling's T2 test).[13, 31, 32 and 33] In tables and figures, the impedance measurements R and Xc are normalized by the subject's height (H) and then expressed as R/H and Xc/H in ohms per meter, in accordance with standard RXc graph methodology.

### DETAILED DISCUSSION OF THE INVENTION

RXc and RXc score graph methods provide bioelectrical impedance vector analysis (BIVA) that is noninvasive, simple, rapid, accurate, and reproducible for determining hydration status (dehydration, volume overload), and nutritional status (obesity, lean muscle mass, cachexia). These methods use simple pattern recognition of confidence intervals of disease populations plotted over a graph of norms. The concept is similar to reading an EKG.

Magnitude and phase angle of individual impedance vectors are utilized for a quick, direct, accurate assessment in health and in disease states both at a point in time and for follow-up. Body composition changes over time following interventions such as diet, exercise, diuretic use, starvation or malnutrition, and progression of disease. Monitoring change over time of body impedance prognosticates survival of individual subjects.

The methods can be applied in dialysis patients for optimizing fluid management and preventing cardiovascular morbidity and mortality attributable to too little fluid (hypotension) or too much fluid (hypertension, pulmonary edema, cardiac stress), in renal patients (both acute and chronic renal failure) to assess fluid status in general, and in heart failure, in planning optimal diuresis. They can be used in the healthy individual such as in climbers to monitor hydration status at high altitude to avoid acute mountain sickness. They can also be used in the athlete to improve performance by monitoring of body composition, nutrition, and hydration. From a nutritional standpoint the graph can be used to follow muscle buildup or breakdown, obesity, cachectic states, etc. Vector angle has been used to prognosticate survival in disease states such as kidney failure, AIDS, anorexia, etc.

The RXc graph method makes three kinds of bioimpedance evaluation possible. These include: (1) Evaluation of a single vector with a length and angle measured the first time in an individual subject, by plotting the point vector on the reference norms (RXc point graph). The tool quickly and accurately assesses and individuals hydration status and relative muscle mass or fat composition at an instant in time. (2). Bioimpedance follow-up: Evaluation of a person's hydration status and relative fat or muscle mass over time after various interventions or over time in disease states, i.e. diet, exercise, starvation malnutrition, volume repletion or depletion (RXc path graph). (3). Evaluation of groups of subjects using the bivariate 95% confidence ellipses of the mean vectors (RXc mean graph). This is useful for clinical and epidemiological research studies aimed to identify disorders in body composition of groups at risk.

The present invention provides, e.g., methods of making and analyzing BIA data using an RXc-score graph. The RXc-score graph is far simpler and more robust than RXc graphs of the prior art. To provide a basis for understanding the RXc-score graph, the following first discusses the RXc graph and then discusses the RXc-score graph, identifying certain differences between them.

The RXc Graph

The RXc graph provides accurate, rapid, reproducible assessment of body composition in terms of relative hydration or volume status and simultaneous assessment of fat and muscle composition by recording resistance (R) and reactance (Xc) each over the patient's height and plotting of the two variables as a point vector on the graph of norms.

Norms are defined as a set of elliptical regions centered on the mean vector, and containing, e.g., 50%, 75%, and 95% of individual vectors measured in the healthy, reference population. The farther a vector is from the mean, the more abnormal the subject's body composition. In adults, vectors falling out of the 75% tolerance ellipse indicate abnormal tissue impedance and body composition. Much like an EKG, one can determine body composition by impedance pattern recognition. BIVA patterns have been derived from clinical validation studies. These include the following.

1. Vector position (steady state) or migration (dynamic state) parallel to the major axis of the tolerance ellipses indicate changes in soft tissue hydration. Dehydration is occurs with vector lengthening out of the upper pole, and hyperhydration with apparent edema occurs with vector shortening out of the lower pole.

2. Vector position (steady state) or migration (dynamic state) parallel to the minor axis that is to the left and up into the left upper quadrant indicates predominance of muscle mass or an athletic individual, and positioning of the vector to the right into the upper right quadrant indicates a lean individual with lower component of muscle mass as compared to the athlete. Migration of the vector to the left indicates an increasing muscle mass, and migration to the right indicates a decrease in muscle mass. Vector positioning in the left lower quadrant indicates an obese subject and movement of the vector into the right lower quadrant indicates a cachectic or severely malnourished individual.

3. Vector displacements along different vector trajectories indicate combined changes in both hydration and tissue mass. In clinical practice, soft tissue hydration can change either rapidly (hours or days) in acute diseases or slowly (chronic diseases) over weeks or months. In contrast, detectable soft tissue mass can change over weeks or months.

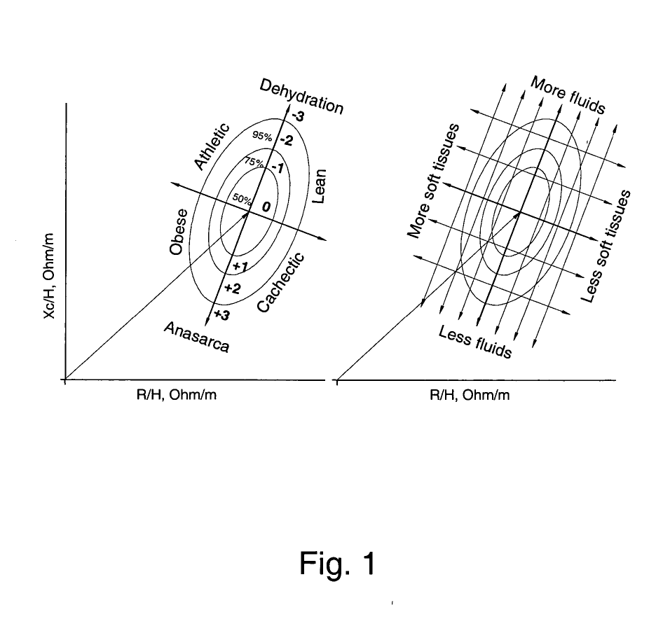

This is further detailed in FIG. 1. As shown in FIG. 1, a forward or backward displacement of vectors parallel to the major axis of ellipses was associated with dehydration or fluid overloading, respectively, reaching extremes out of the poles. Vectors above or below the major axis (meaning upper-left or lower-right half of ellipses) were associated with more or less cell mass in soft tissues, respectively, with extremes along the minor axis.

On the RXc graph, a vector position is compared with a set of three tolerance ellipses (e.g., 50%, 75%, and 95%) calculated in a specific reference population (e.g., with a fixed gender, race, age and BMI class). The shape of the tolerance ellipse is determined by the value of the correlation coefficient between R and Xc.

Therefore, one RXc graph with three tolerance ellipses are drawn for every specific reference population. As with other medical devices, measurements are best achieved with the same type of impedance analyzer in the new individuals and in the reference population.

The RXc Z-Score Graph

The RXc Z-score graph expands upon the concepts of the RXc graph, leading to a generalized graphical method using statistical standardization of both R and Xc. In the RXc Z-score graph method, R and Xc measurements become bivariate Z scores that are centered, e.g., on the zero mean, with unit standard deviation, and range e.g., between 0 and ±3. A statistical Z-score is defined as an individual value minus the mean and divided by the standard deviation of a reference population. Hence, a Z-score is a pure, dimensionless number that expresses the distance of an individual value from the mean in terms of the standard deviation.

The RXc-score graph is unique for all subjects, and is formed with three tolerance ellipses (e.g., 50%, 75%, and 95%) centered on the value 0 of both standardized vector components as Z(R) and Z(Xc). A further advantage to the z-score graph as opposed to the RXc graph is that bivariate Z-scores utilize R and Xc instead of R/H and Xc/H, simplifying the entire procedure (described in further detail below). The shape of tolerance ellipses is determined by the correlation coefficient between Z(R) and Z(Xc). The universal correlation coefficient for the RXc z-score graph was calculated on 8,022 healthy subjects of different races, genders, body mass indices, and ages, participating in the NHANES III study (Third National Health and Nutrition Examination Survey). This is described in more detail in Piccoli, Pillon and Dumler (2002) “Impedance vector distribution by sex, race, body mass index, and age in the United States: standard reference intervals as bivariate Z scores” Nutrition 18:153-167; See also, Appendix 1.

In practice, Z scores, i.e. Z(R) and Z(Xc), are calculated for impedance readings R and Xc with respect to the mean and standard deviation of the reference population, and then are plotted on the Z-score graph. One is now able to identify the BIVA patterns as with the original RXc graph, but independent of the person's gender, race, body mass index, age, and independent on the bioimpedance analyzer used.

Finally, the RXc-z score graph allows comparison of results from literature that were obtained in different populations and with different analyzers.

BIVA patterns from groups of individuals with different hydration or body composition from literature, plotted on the RXc z-score graph illustrate the practical application of the graph.

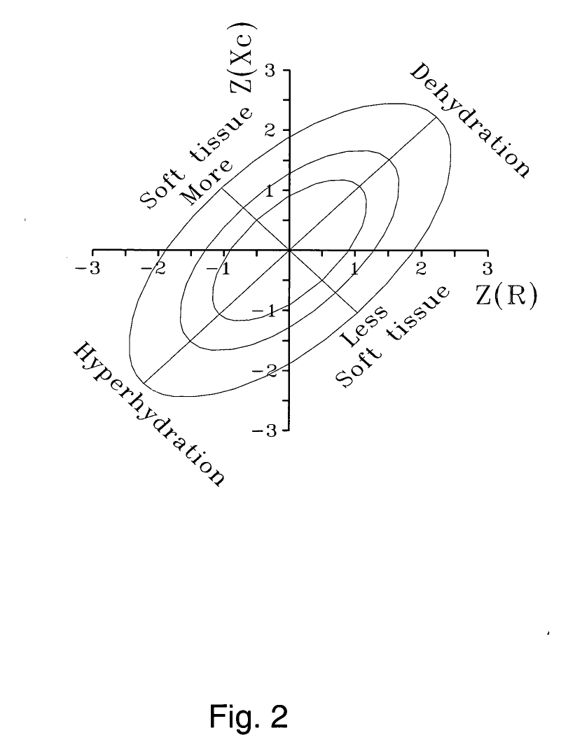

Data in FIG. 2 are drawn from the literature and plotted on the RXc z-score graph after transformation of impedance measurements from several disease groups into bivariate Z scores (additional details below).

Solid and open circles represent male and female gender, respectively.

Single vectors are from athletes, obese subjects of class I to III (Ob/1-3) or class I (Ob/1) patients with chronic renal failure having nephrotic syndrome (Edema), lung cancer (cancer), AIDS in stage WR 3-5 (HIV/3-5) or WR 6 (HIV/6), and anorexia nervosa.

Repeated score vectors are from climbers before and after high altitude dehydration, hemodialysis (HD) patients, either lean or obese (HDo) before and after fluid removal with a dialysis session, and dehydrated patients with cholera before and after fluid infusion.

RXc-Score Graph on Bivariate Z Scores

Because the size and shape of tolerance ellipses of the sex- and race-specific NHANES III reference populations were comparable despite different mean values, only one RXc-score graph could be derived to represent, with a good approximation, reference intervals for any individual from these subpopulations (FIG. 2). In a bivariate Z-score system, deviations from the means of both components are centered on zero and can be scaled between +3 and −3, which also determines fixed slopes of major (+1) and minor (−1) axes of standardized ellipses (FIG. 2). Therefore, the construction of the RXc-score graph was sensitive only to the correlation coefficient between vector components. In pooled NHANES III reference populations, the correlation coefficients (r) between R/H and Xc/H were 0.631 in 4226 men and 0.645 in 3796 women, on average 0.638 in 8022 subjects, with a 95% confidence interval (0.60 to 0.68) that included r fluctuations of subpopulations (Table V). Therefore, we constructed the reference RXc-score graph using r=0.638, which determined the shape of the 50%, 75%, and 95% tolerance ellipses centered on the zero mean vector (FIG. 2).

As shown in FIG. 2, the reference RXc-score graph with 95%, 75%, and 50% tolerance ellipses. Both dimensionless Z scores, i.e., resistance score Z(R) and reactance score Z(Xc), are the difference of individual R and Xc values from the mean divided by the standard deviation of the reference population. Drawing parameters of ellipses are reported in Table VI. R, resistance; Xc, reactance.

Interestingly, differences between bivariate Z scores calculated on pairs of R/H and Xc/H versus R and Xc were negligible (<0.1 SD) because individual statures were close to the mean stature of the sex- and race-specific reference population. In practice, therefore, either pair of impedance vector deviates can be plotted on the same RXc-score graph (i.e., on very close loci), where dimensionless abscissa and ordinate axes are indicated as Z(R) and Z(Xc), respectively (FIG. 2).

Plotting individual impedance vector deviates on the RXc-score graph (on a dimensionless scale) allows classification of a subject into the same four categories as with the original impedance vector on the RXc graph (/m scale), i.e., falling within the median, 3rd quartile, or 95th percentile, or outside the reference intervals.

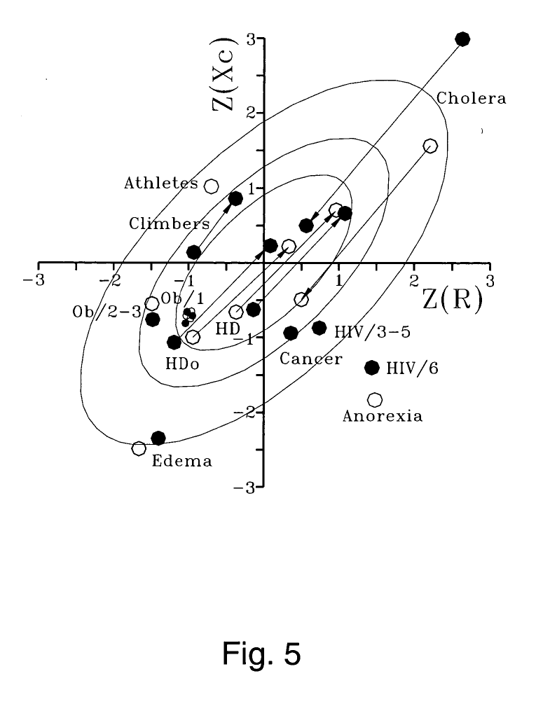

In FIG. 5 we plotted on the RXc-score graph several mean vector scores obtained from the literature after transformation of original impedance measurements into standard deviates with respect to the mean and SD of their reference populations. As shown in FIG. 5, data drawn from the literature and plotted on the RXc-score graph after transformation of impedance measurements from several disease groups into bivariate Z scores (with respect to their reference population). Solid and open circles represent male and female, respectively. Single score vectors are from athletes,[41] obese subjects of class I to III [18] or class I (NHANES III population, this study), patients with CRF in conservative treatment, [13] nephrotic syndrome (edema), [18] lung cancer, [40] acquired immunodeficiency syndrome in stages WR 3 to 5 or WR 6, [42] and anorexia nervosa. [43] Repeated score vectors are from climbers before and after high altitude dehydration, [15] HD patients, either lean [16] or obese, [18] before and after fluid removal with a dialysis session, and dehydrated patients with cholera before and after fluid infusion. [44] A forward or backward displacement of vectors parallel to the major axis of ellipses was associated with dehydration or fluid overloading, respectively, reaching extremes out of the poles. Vectors above or below the major axis (meaning upper left or lower right half of ellipses) were associated with more or less cell mass in soft tissues, respectively, with extremes along the minor axis. CRF, chronic renal failure; HD, hemodialysis; HDo, obese hemodialysis patients; HIV, human immunodeficiency virus; HIV/3-6, stages WR 1-6; Ob/1-3, obese subjects of classes I to III.

Formulas for Calculating Confidence and Tolerance Ellipses for the RXc Graph and the RXC-Score Graph

In the case of bivariate normal distribution, confidence and tolerance intervals can be calculated by exact methods.[31 and 33] After suitable modification of formulas, common statistics of simple linear correlation analysis can be used for calculations. [13] Given n pairs of observations x and y, with SDs sx and sy, and a correlation coefficient r, for a fixed probability level, take the Snedecor's F value with 2 and n−2 degrees of freedom. The semiaxes L1 and L2 and the slopes b1 and b2=−1/b1 of the axes of the 100(1-) % confidence and tolerance ellipses (e.g., =0.05, 0.25, and 0.50 for the 95th, 75th, and 50th percentiles, respectively) of RXc graphs can be calculated using (1a) and (2a), respectively. Parameters of tolerance ellipses of bivariate Z scores (RXc-score graph) can be calculated accordingly using (1b) and (2b).

Accordingly: given n pairs of observations x and y, with standard deviation sₓ and s_{y}, and correlation coefficient r, for a fixed α probability level, take the Snedecor's F_{α} value with 2 and n−2 degrees of freedom.

RXc Graph

The semi-axes L₁ and L₂ and the slopes b₁ and b₂=−1/b₁, of the axes of the 100(1-α) % confidence and tolerance ellipses (e.g. α=0.05, 0.25, and 0.50 for the 95^{th}, 75^{th}, and 50^{th} percentile, respectively) of RXc graphs can be calculated using equations (1a) and (2a), respectively.

RXc Z-Score Graph

Parameters of tolerance ellipses of bivariate Z scores (RXc-score graph) can be calculated accordingly, using equations (1b) and (2b).

$${L}_{1} , {L}_{2} = √ K \cdot \sqrt{\begin{matrix}( n - 1 ) ( {s}_{x}^{2} + {s}_{y}^{2} ) \pm \\ \sqrt{{[ ( n - 1 ) ( {s}_{x}^{2} + {s}_{y}^{2} ) ]}^{2} - 4 {( n - 1 )}^{2} ( 1 - {r}^{2} ) {s}_{x}^{2} {s}_{y}^{2}}\end{matrix}} \qquad ( Eq . 1 a )$$

L₁,L₂=√K·√2(n−1)±2r(n−1) (Eq. 1b)

where

K=F/n·(n−2) for confidence ellipses

K=F·(n+1)/n·(n−2) for tolerance ellipses

b₁,b₂=(b,−1/b)=(s_{y}²−sₓ²)/2rsₓs_{y}±√1+[(s_{y}²−sₓ²)/2rsₓs_{y}]² (Eq. 2a)

b₁,b₂=±1 (Eq. 2b)

Software for the RXc graphs and vector BIA is available from Antonio Piccoli (apiccoli@unipd.it).

Example BIVA Software 2002

BIVA confidence and BIVA tolerance are optionally represented in two Excel files (Visual Basic macros) for the Bioelectrical Impedance Vector Analysis (BIVA) with the RXc Graph and the RXc-score graph.

BIVA Tolerance File

Three tolerance ellipses are drawn corresponding to the 50th, 75th and 95th percentile of the impedance vector distribution of a selected, reference population.

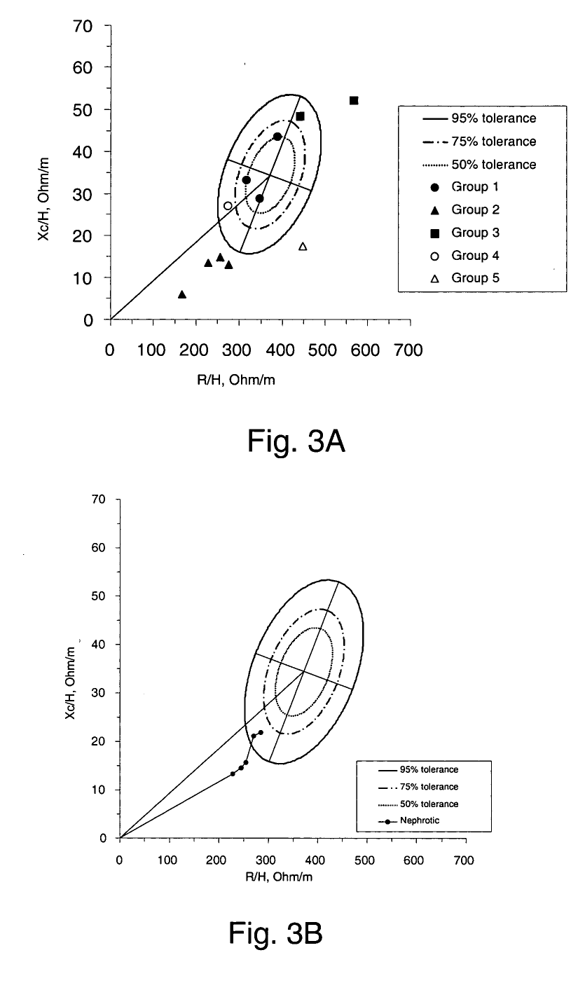

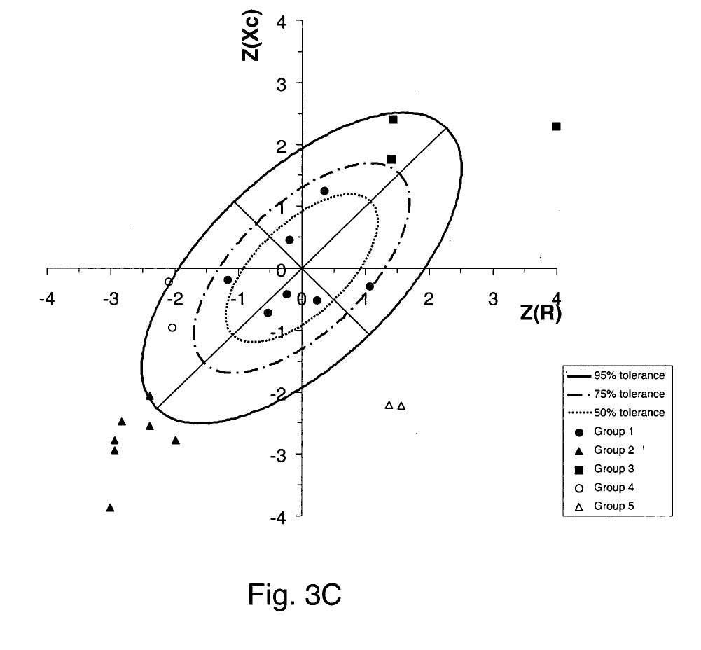

Options for vector analysis with BIVA tolerance: 1. Plot of one point vector for every subject on the tolerance ellipses (RX point graph). 2. Plot of a sequence of point vectors for one subject on the tolerance ellipses (RXc path graph). 3. Plot of one bivariate Z-score vector for every subject on the reference RXc-score graph with three universal, tolerance ellipses (50%, 75%, and 95%). (FIG. 3, Panels A-C).

Z-Score Graph

The 50%, 75%, and 95% tolerance ellipses are drawn for the standard, reference RXc-score graph representing the bivariate distribution of the standard deviates of R/H and Xc/H with zero mean, unit standard deviation, and their correlation coefficient r=0.638 (NHANES III estimates on 8022 adults) according to the RXc-score graph methodology [23] (formulas can be found in Piccoli, Pillon and Dumler (2002) “Impedance vector distribution by sex, race, body mass index, and age in the United States: standard reference intervals as bivariate Z scores” Nutrition 18:153-167). See also, Appendix 1.

The two standard deviates of the bivariate Z-score vector are calculated from individual R/H and Xc/H data as Z(R)=(R/H—the mean value)/SD, and Z(Xc)=(Xc/H—the mean value)/SD, that is with respect to the means and standard deviations of a specific, reference population by race, gender, BMI range, age range, and analyzer.

The individual impedance vector deviates are plotted on the RXc-score graph (dimensionless scale) allowing classification of a subject into the same categories as with the original impedance vector on the RXc-graph (Ohm/m scale), i.e. falling within the median, 3^{rd} quartile, or 95^{th} percentile, or outside the reference intervals.

Different symbols by group of subjects are used up to 10 classification groups (codes 1 to 10 are recorded in the Group code column in the Z-score sheet) whose score vectors are plotted as points on the RXc-score graph.

BIVA Confidence File

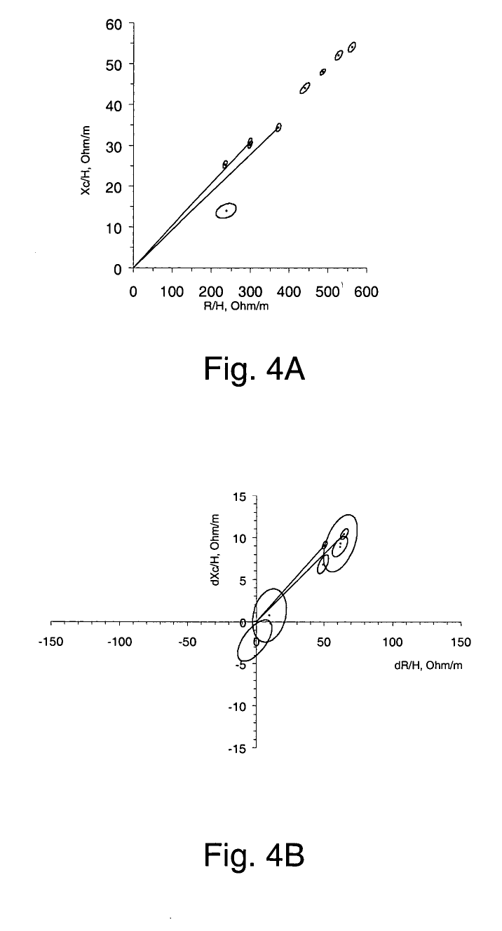

Unpaired data analysis. The 95% confidence ellipses are drawn for the mean impedance vectors of 1 to 128 groups. FIG. 4, Panel A.

Paired data analysis. Mean vectors can also be mean vector displacements which are differences (e.g. post minus pre values, or pre minus post values) of both vector components. FIG. 4, Panel B.

Options for Vector Analysis with BIVA Confidence:

1. Plot of mean vectors with the 95% confidence ellipse for every group of subjects (RXc mean graph). Separate 95% confidence ellipses are equivalent to a statistically significant (P<0.05) two-sample Hotelling's T² test.

2. Plot of the mean difference vector (vector displacement) with the 95% confidence ellipse of the difference for every group of subjects (Paired RXc mean graph). If a 95% confidence ellipse of a vector displacement does not cover the origin of the dRXc graph, then the vector displacement is statistically significant (P<0.05), which is equivalent to a significant paired one-sample Hotelling's T² test.

Systems for Practicing the Methods

The systems of the invention can include system instructions for practicing any of the methods herein, e.g., generated by or embodied in system software. The systems typically include a user viewable display that permits a user to view data, e.g., BIA data, e.g., plotted on an RXc or RXc-score graph. For example, the system can include a computer that comprises a user viewable display and the system software. Example system software is available from Antonio Piccoli (apiccoli@unipd.it).

Optionally, the system can further include components that take the relevant R or Xc measurements, e.g., a plethesmograph or other device comprising appropriate electrical leads configured to be placed on the skin of a patient. The components are operably coupled to the other system elements, e.g., via cables or wireless connections (e.g., IR connections). For example, the R or Xc measurement components can be coupled to a computer comprising the relevant system instructions and display.

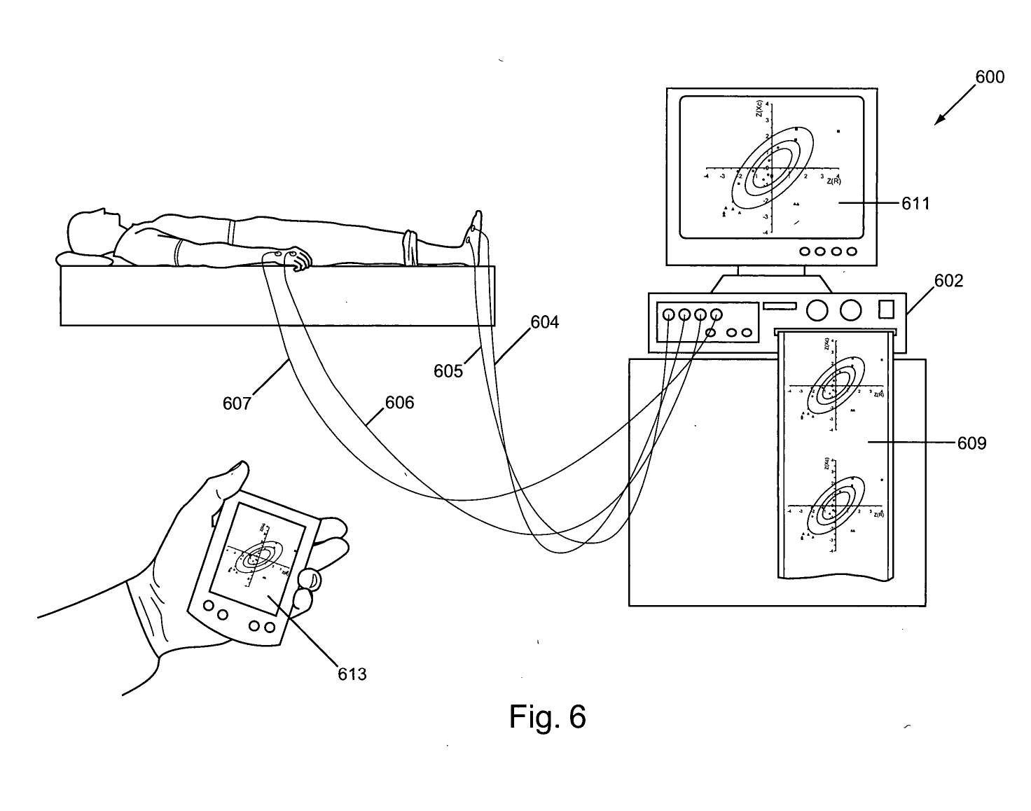

An example system is illustrated in FIG. 6. As shown, system 600 comprises plethesmograph 602 which can be coupled to a patient through leads 604-607. In one typical embodiment, as depicted, two leads are coupled to the top of the foot and two leads are coupled to the back of the hand, per standard procedures for plethesmography. In the depicted embodiment, plethesmograph 602 comprises appropriate system software for producing an RXc-Z score graph and placing plethesmography readings from the patient onto the graph as described herein. In alternate embodiments, the system comprises one or more separate computers that comprise such software, in operable linkage with plethesmograph 602. The graphical readout can be in the form of a print readount, e.g., print display 609, a monitor, e.g., display 611, or a handheld display, e.g., hand-held display 613. Any of these displays can be coupled to plethesmograph 602 through appropriate operable linkages, e.g., cables, infrared linkages, or the like.

### EXAMPLES

The following examples are not limiting. One of skill will immediately recognize parameters that can be changed without substantially altering the scope of the claims.

Bioelectrical impedance measurements were collected in the Third National Health and Nutrition Examination Survey (NHANES III), but their results have not been published. In the NHANES III population, resistance (R) and reactance (Xc) values at 50-kHz frequency were obtained with a Valhalla Scientific meter (model 1990B; San Diego, Calif., USA). The RXc graph method was used to identify bivariate pattern distributions of mean vectors (95% confidence ellipses by sex, race, age, and body mass index [BMI]), and individual impedance vectors (50%, 75%, and 95% tolerance ellipses). Data from 10 222 adults (5261 men and 4961 women) formed 90 four-way classification groups, with two sexes, three races or ethnicities (non-Hispanic white, non-Hispanic black, Mexican American), five age classes (20-29, 30-39, 40-49, 50-59, and 60-69 y), and three BMI classes (19-24.9, 25-29.9, and 30-34.9 kg/m2). Sex, race or ethnicity, BMI and age, in decreasing order, influenced the vector distribution pattern. Mean vectors in women were significantly longer than those in men. Within each sex, the mean vector of non-Hispanic white subjects was shorter and with a smaller phase angle than that of corresponding BMIs from the two other race/ethnic populations. Tolerance ellipses were calculated from sex- and race-specific reference populations 20 to 69 y old and 19 BMI<30 kg/m2 (8022 subjects, 4226 men and 3796 women). After transformation of impedance vector components into bivariate Z scores (standardized deviates, as differences from the mean divided by the standard deviation of the reference population), we constructed one standard, reference, RXc-score graph (50%, 75%, and 95% tolerance ellipses) that can be used with any analyzer in any population. The pattern of impedance vector distribution and reference bivariate intervals for the individual impedance vector are presented for comparative studies (software available at E-mail: apiccoli@unipd.it).

Complete details on the above are found in Piccoli and Pillon (2002) “Impedance vector distribution by sex, race, body mass index, and age in the United States: standard reference intervals as bivariate Z scores” Nutrition 18:153-167, incorporated herein by reference in its entirety. See also, Appendix 1.

### REFERENCE

1. R. J. Kuczmarski, K. M. Flegal, S. M. Campbell and C. L. Johnson, Increasing prevalence of overweight among US adults. The National Health and Nutrition Examination Surveys. 1960 to 1991. JAMA 272 (1994), p. 205. Abstract-EMBASE|Abstract-MEDLINE

2. K. M. Flegal, M. D. Carrol, R. J. Kuczmarski and C. L. Johnson, Overweight and obesity in the United States: prevalence and trends, 1960-1994. Int J Obes 22 (1998), p. 39. Abstract-EMBASE|Abstract-MEDLINE

3. Plan and operation of the third National Health and Nutrition Examination Survey, 1988-1994. Series 1, 32. DHHS publication (PHS) 94 1308, US Government Printing Office, Washington, D.C. (1994).

4. National Center for Health StatisticsHealth. United States, 1995, Public Health Service, Hyattsville, Md. (1996).

5. R. J. Kuczmarski, Bioelectrical impedance analysis measurements as part of a national nutrition survey. Am J Clin Nutr 64 suppl (1996), p. 453S. Abstract-MEDLINE|Abstract-Elsevier BIOBASE

6. R. J. Kuczmarski, M. D. Carroll, K. M. Flegal and R. P. Troiano, Varying body mass index cutoff points to describe overweight prevalence among U.S. adults: NHANES III (1988 to 1994). Obes Res 5 (1997), p. 542. Abstract-MEDLINE

7. WHO Expert Committee on Physical Status The use and interpretation of anthropometry. Technical Report 854, World Health Organization, Geneva (1995).

8. J. G. Meisler and S. S. Jeor, Summary and recommendations from the American Health Foundation's expert panel on healthy weight. Am J Clin Nutr 63 suppl (1996), p. 474S. Abstract-MEDLINE|Abstract-EMBASE

9. US Department of Agriculture, Agricultural Research Service, Dietary Guidelines Advisory Committee Report of the Dietary Guidelines Advisory Committee on the Dietary Guidelines for Americans, 1995, to the Secretary of Health and Human Services and the Secretary of Agriculture, US Department of Agriculture, Beltsville, Md. (1995).

10. International Obesity Task Force Obesity: preventing and managing the global epidemic: report of a WHO Consultation on Obesity, Geneva, 3-5 Jun., 1997, World Health Organization, Geneva (1998).

11. H. C. Lukaski, W. W. Bolonchuck, C. B. Hall and W. A. Siders, Validation of tetrapolar bioelectrical impedance method to assess human body composition. J Appl Physiol 60 (1986), p. 1327. Abstract-EMBASE|Abstract-MEDLINE

12. L. B. Houtkooper, T. G. Lohman, S. B. Going and W. H. Howell, Why bioelectrical impedance analysis should be used for estimating adiposity. Am J Clin Nutr 64 suppl (1996), p. 436S. Abstract-Elsevier BIOBASE|Abstract-MEDLINE

13. A. Piccoli, B. Rossi, L. Pillon and G. Bucciante, A new method for monitoring body fluid variation by bioimpedance analysis: the RXc graph. Kidney Int 46 (1994), p. 534. Abstract-MEDLINE|Abstract-EMBASE

14. A. Piccoli, S. Nigrelli, A. Caberlotto et al., Bivariate normal values of the bioelectrical impedance vector in adult and elderly populations. Am J Clin Nutr 61 (1995), p. 269. Abstract-MEDLINE

15. A. Piccoli, P. Piazza, D. Noventa, L. Pillon and M. Zaccaria, A new method for monitoring hydration at high altitude by bioimpedance analysis. Med Sci Sports Exerc 28 (1996), p. 1517. Abstract-MEDLINE|Abstract-EMBASE

16. Italian HD-BIA Study GroupA. Piccoli, Identification of operational clues to dry weight prescription in hemodialysis using bioimpedance vector analysis. Kidney Int 53 (1998), p. 1036. Abstract-EMBASE|Abstract-MEDLINE

17. A. Piccoli, G. Pittoni, E. Facco, E. Favaro and L. Pillon, Relationship between central venous pressure and bioimpedance vector analysis in critically ill patients. Crit Care Med 28 (2000), p. 132. Abstract-MEDLINE|Abstract-EMBASE

18. A. Piccoli, A. Brunani, G. Savia et al., Discriminating between body fat and fluid changes in the obese adult using bioimpedance vector analysis. Int J Obes 22 (1998), p. 97. Abstract-EMBASE|Abstract-MEDLINE

19. H. C. Lukaski, A new approach to estimate changes in total body water by bioelectrical impedance analysis. Nutrition 13 (1997), p. 474. Abstract-MEDLINE

20. G. M. Chertow, J. M. Lazarus, N. L. Lew, L. Ma and E. G. Lowrie, Biompedance norms for the hemodialysis population. Kidney Int 52 (1997), p. 1617. Abstract-EMBASE|Abstract-MEDLINE

21. G. M. Chertow, D. O. Jacobs, J. M. Lazarus et al., Phase angle predicts survival in hemodialysis patients. J Renal Nutr 7 (1997), p. 204.

22. T. A. Ikizler, R. L. Wingard, J. Harvell, Y. Shyr and M. Hakim, Association of morbidity with markers of nutrition and inflammation in chronic hemodialysis patients: a prospective study. Kidney Int 55 (1999), p. 1945. Abstract-EMBASE|Abstract-MEDLINE|Full Text via CrossRef

23. A. Piccoli, L. Pillon and M. G. Tabbì, Major confounders for reactance as a marker of malnutrition in hemodialysis patients. Kidney Int 56 (1999), p. 2311. Abstract-MEDLINE|Abstract-EMBASE|Abstract-Elsevier BIOBASE|Full Text via CrossRef

24. B. DiIorio and V. Bellizzi, Association of mortality and morbidity with bioimpedance analysis. Kidney Int 58 (2000), p. 2512.

25. B. Guida, L. De Nicola, R. Trio et al., Comparison of vector and conventional bioelectrical impedance analysis in the optimal dry weight prescription in hemodialysis. Am J Nephrol 20 (2000), p. 311. Abstract-EMBASE|Abstract-MEDLINE|Full Text via CrossRef

26. R. M. Ezzati, J. T. Massey, J. Waksberg, A. Chu and K. R. Mauer, Sample design: third National Health and Nutrition Examination Survey. Vital Health Stat 2 (1992), p. 113.

27. US Department of Health and Human Services (DHHS)National Center for Health StatisticsThird National Health and Nutrition Examination Survey, 1988-1994, NHANES III Laboratory Data File (CD-ROM). Public Use Data File Documentation Number 76200, Centers for Disease Control and Prevention, Hyattsville, Md. (1996).

28. US Department of Health and Human Services (DHHS)National Center for Health StatisticsNHANES III Reference manuals and Reports (CD-ROM), Centers for Disease Control and Prevention, Hyattsville, Md. (1996).

29. C. A. Jones, G. M. McQuillan, J. W. Kusek et al., Serum creatinine levels in the US population: third national health and nutrition examination survey. Am J Kidney Dis 32 (1998), p. 992. Abstract-EMBASE|Abstract-MEDLINE

30. SAS InstituteSAS procedure guide, version 6 (3rd ed.), SAS Institute, Cary, N.C. (1990).

31. C. Lentner, Introduction to statistics. Statistical tables. Mathematical formulae. In: Geigy scientific tables, Vol 2 (8th ed.), Ciba-Geigy Limited, Basel (1982), p. 215.

32. E. K. Harris and J. C. Boyd Statistical bases of reference values in laboratory medicine, Marcel Dekker, N.Y. (1995).

33. P. Jolicoeur Introduction to biometry, Kluwer Academic/Plenum Publishers, New York (1999).

34. L. C. Ward and B. L. Heitmann, Electrical maturation trajectory of human tissues identified by bioelectrical impedance vector analysis. Nutrition 16 (2000), p. 319. Abstract-MEDLINE|Abstract-EMBASE

35. C. Pichard, U. G. Kyle, D. Bracco et al., Reference values of fat-free and fat masses by bioelectrical impedance analysis in 3393 healthy subjects. Nutrition 16 (2000), p. 245. SummaryPlus|Full Text+Links|PDF (169 K)

36. L. C. Ward, B. L. Heitmann, P. Craig et al., Association between ethnicity, body mass index, and bioelectrical impedance. Ann NY Acad Sci 904 (2000), p. 199. Abstract-MEDLINE|Abstract-EMBASE

37. M. F. Kuczmarski, R. J. Kuczmarski and M. Najjar, Descriptive anthropometric reference data for older Americans. J Am Diet Assoc 100 (2000), p. 59. Abstract-MEDLINE|Abstract-EMBASE

38. A. Piccoli, L. Pillon and E. Favaro, Asymmetry of the total body water prediction bias using the impedance index. Nutrition 13 (1997), p. 438. Abstract-EMBASE|Abstract-MEDLINE

39. N. M. Oldham, Overview of bioelectrical impedance analyzers. Am J Clin Nutr 64 suppl (1996), p. 405S. Abstract-Elsevier BIOBASE|Abstract-MEDLINE

40. S. Toso, A. Piccoli, M. Gusella et al., Altered tissue electric properties in lung cancer patients as detected by bioelectric impedance vector analysis. Nutrition 16 (2000), p. 120. SummaryPlus|Full Text+Links|PDF (117 K)

41. H. C. Lukaski, W. W. Bolonchuk, W. A. Siders and C. B. Hall, Body composition assessment of athletes using bioelectrical impedance measurements. J Sports Med Phys Fitness 30 (1990), p. 434. Abstract-EMBASE|Abstract-MEDLINE

42. M. Ott, B. Lembcke, H. Fischer et al., Early changes of body composition in human immunodeficiency virus-infected patients: tetrapolar impedance analysis indicates significant malnutrition. Am J Clin Nutr 57 (1993), p. 15. Abstract-EMBASE|Abstract-MEDLINE

43. L. Scalfi, G. Di Biase, C. Sapio, A. Coltorti and F. Contaldo, Bioimpedance analysis and resting energy expenditure in undernourished and refed anorectic patients. Eur J Clin Nutr 47 (1993), p. 61. Abstract-MEDLINE|Abstract-EMBASE

44. J. J. McDonald, B. Chanduvi, G. Velarde et al., Bioimpedance monitoring of rehydration in cholera. Lancet 341 (1993), p. 1049. Abstract-EMBASE|Abstract-Medline

While the foregoing invention has been described in some detail for purposes of clarity and understanding, it will be clear to one skilled in the art from a reading of this disclosure that various changes in form and detail can be made without departing from the true scope of the invention. For example, all the techniques and systems described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of bioelectrical impedance vector analysis, the method comprising:
determining Z(R) and Z(Xc) for a patient; and,
plotting Z(R) and Z(Xc) on an RXc Z-score graph.

2. The method of claim 1, wherein Z(R) and Z(Xc) are determined by taking impedance readings for R and Xc for the patient and subtracting a mean value for R and Xc, respectively, for a reference population, from the impedance readings R and Xc, respectively, and dividing the resulting values by the standard deviation for R and Xc, respectively, in the reference population.

3. The method of claim 2, wherein R and Xc are measured on an impedance plethysmograph.

4. The method of claim 3, wherein the impedance plethysmograph is a single frequency plethysmograph.

5. The method of claim 3, wherein the impedance plethysmograph is a multi-frequency plethysmograph.

6. The method of claim 2, wherein the magnitude of the impedance is equal to the square root of (R²+Xc²) and the impedance has a phase angle equal to arctan Xc/R and a length equal to the square root of (R²+Xc²).

7. The method of claim 1, wherein the method comprises a whole body impedance measurement.

8. The method of claim 1, wherein the method comprises an impedance measurement of a portion of a whole body.

9. The method of claim 1, wherein the RXc Z-score graph comprises one or more ellipse showing deviation from an absolute mean for a patient population.

10. The method of claim 1, wherein the RXc Z-score graph comprises three tolerence ellipses showing deviation from an absolute mean for a patient population at 50%, 75% and 95% deviation from mean.

11. The method of claim 9, wherein placement of Z(R) and Z(Xc) for the patient on the graph provides diagnostic information for the patient.

12. The method of claim 1, wherein plotting Z(R) and Z(Xc) on the RXc Z-score graph provides an indication of whether the patient has more or less soft tissue or is more or less hydrated than a mean for a reference population.

13. The method of claim 12, wherein the indication is correlated to one or more of: leanness, obesity, dehydration, anasarca, whether the patient is athletic, whether the patient is cachectic, whether the patient is normal, or any combination thereof.

14. A method of bioelectrical impedance vector analysis, the method comprising:
determining R and Xc for a patient;
converting R and Xc into dimensionless numbers; and,
plotting the resulting dimensionless numbers on a graph.

15. The method of claim 14, wherein R and Xc are determined by measuring them on an impedance plethysmograph.

16. The method of claim 14, wherein R and Xc are converted into dimensionless numbers by subtracting a population mean for R and Xc, respectively, from R and Xc, respectively, and dividing the resulting number by the standard deviation for R and Xc, respectively, in the population.

17. The method of claim 14, wherein the graph comprises one or more standardized ellipse showing deviation from an absolute mean of a dimensionless number for R and Xc for a patient population.

18. The method of claim 14, wherein the graph comprises three tolerence ellipses showing deviation from an absolute mean for a patient population at 50%, 75% and 95% deviation from mean.

19. The method of claim 14, wherein the graph of standardized dimensionless numbers is an RXc Z-score graph.

20. The method of claim 19, wherein plotting the dimensionless numbers for R and Xc on the RXc Z-score graph provides an indication of whether the patient has more or less soft tissue or is more or less hydrated than a mean for a reference population.

21. The method of claim 20, wherein the indication is correlated to one or more of: leanness, obesity, dehydration, anasarca, whether the patient is athletic, whether the patient is cachectic, whether the patient is normal, or any combination thereof.

22. A method of performing a graphical bioelectrical impedance vector analysis, the method comprising:
plotting Z(Xc) versus Z(R) for one or more patient or patient population; and,
viewing the resulting Z(Xc)/Z(R) graph.

23. The method of claim 22, wherein Z(Xc) versus Z(R) is plotted by a computer.

24. The method of claim 22, wherein Z(R) and Z(Xc) are determined for the patient or population by taking impedance readings for R and Xc for the patient or the population and subtracting a mean value for R and Xc, respectively, for a reference population, from the impedance readings R and Xc, respectively, and dividing the resulting values by the standard deviation for R and Xc, respectively, in the reference population.

25. The method of claim 24, wherein R and Xc are measured on an impedance plethysmograph.

26. The method of claim 22, wherein plotting Z(R) and Z(Xc) on the RXc Z-score graph provides an indication of whether the patient has more or less soft tissue or is more or less hydrated than a mean for a reference population.

27. The method of claim 26, wherein the indication is correlated to one or more of: leanness, obesity, dehydration, anasarca, whether the patient is athletic, whether the patient is cachectic, whether the patient is normal, or any combination thereof.

28. The method of claim 22, wherein the graph is viewed on a user-viewable display operably coupled to a computer.

29. A method of bioelectrical impedance vector analysis, the method comprising:
determining a dimensionless measurement for R and a dimensionless measurement for Xc; and,
plotting the resulting dimensionless measurement for R against the dimensionless measurement for Xc.

30. The method of claim 29, wherein R and Xc are determined by measuring them on an impedance plethysmograph prior to said determining step.

31. The method of claim 29, wherein the determining comprises converting R and Xc into dimensionless measurements by subtracting a population mean for R and Xc, respectively, from R and Xc, respectively, and dividing the resulting number by the standard deviation for R and Xc, respectively, in the population.

32. The method of claim 29, wherein the dimensionless measurements are plotted against each other using a computer.

33. The method of claim 22, wherein the graph is viewed on a user-viewable display operably coupled to a computer.

34. A bioelectrical impedance vector analysis system comprising an RXc Z-score graph and a user-viewable display displaying the RXc Z-score graph.

35. The system of claim 34, wherein the RXc Z score graph is generated by or embodied in system software.

36. The system of claim 34, wherein the system comprises a computer comprising system software that correlates the RXc Z score graph to whether a patient coupled to the system is one or more of: lean, obese, suffering from dehydration, suffering from anasarca, athletic, cachectic, or normal.

37. The system of claim 34, further comprising electrical leads configured to be placed in contact with the skin of a patient.

38. A bioelectrical impedance vector analysis system comprising a graph of a measure of Xc versus a measure of R, wherein Xc and R are dimensionless; and,
a user-viewable display displaying the graph.

39. The system of claim 38, wherein the graph is an RXc Z-score graph.

40. The system of claim 38, wherein the graph is generated by or embodied in system software.

41. The system of claim 38, wherein the system comprises a computer comprising system software that correlates the graph to whether a patient coupled to the system is one or more of: lean, obese, suffering from dehydration, suffering from anasarca, athletic, cachectic, or normal.

42. The system of claim 38, further comprising electrical leads configured to be placed in contact with the skin of a patient.
